(19) United States
(12) Patent Application Publication
(10) Pub. No.: US 2024/0293599 A1
(43) Pub. Date: Sep. 5, 2024

(54) BONE-REGENERATION-PROMOTING MATERIAL, AND METHOD FOR PRODUCING BONE-REGENERATION-PROMOTING MATERIAL

(71) Applicant: TOHOKU UNIVERSITY, Sendai-shi (JP)

(72) Inventors: Osamu SUZUKI, Sendai-shi (JP); Ryo HAMAI, Sendai-shi (JP); Susumu SAKAI, Sendai-shi (JP); Kaori TSUCHIYA, Sendai-shi (JP)

(21) Appl. No.: 18/663,231

(22) Filed: May 14, 2024

Related U.S. Application Data
(63) Continuation-in-part of application No. PCT/JP2022/043150, filed on Nov. 22, 2022.

(30) Foreign Application Priority Data
Nov. 24, 2021 (JP) 2021-190558

(51) Int. Cl. A61L 27/12; A61L 27/22

(52) U.S. Cl. CPC A61L 27/12; A61L 27/222; A61L 2430/02

(57) ABSTRACT

A bone-regeneration-promoting material containing a composite of octacalcium phosphate and a bioabsorbable polymer and having edge dislocations introduced in the octacalcium phosphate in which the total dislocation density of the octacalcium phosphate is 0.30×10¹⁷ m⁻² or more.

### CROSS-REFERENCE TO RELATED APPLICATIONS

This application is a continuation-in-part of and claims the right of priority to International Application No. PCT/JP2022/043150, filed on Nov. 22, 2022, which, in turn, claims priority to Japanese Patent Application No. 2021-190558, filed Nov. 24, 2021, the disclosures of which are hereby incorporated by reference herein in their entireties for all purposes.

### TECHNICAL FIELD

The present invention relates to a bone-regeneration-promoting material and a production method thereof.

### BACKGROUND ART

Bone grafting is generally used for patients with a bone defect following surgery of a bone tumor, cheilognathopalatoschisis, a comminuted fracture, or the like. Bone grafting is sometimes used also for repairing a bone defect resulting from surgery in the areas of brain surgery, orthopedics, dentistry, and the like.

For bone grafting, an autologous bone is preferably used. To use an autologous bone, however, there is a limit on the amount, and there is a problem such as the damage that remains after the autologous bone is taken out. Thus, an artificial bone which can replace the autologous bone has been developed as a bone used for bone grafting.

As artificial bone materials, hydroxyapatite (Ca₁₀(PO₄)₆(OH)₂: sometimes referred to as “HA” below) ceramics, β-tricalcium phosphate (β-TCP) ceramics, and the like have been proposed.

It is known that a precursor of HA, octacalcium phosphate (Ca₈H₂(PO₄)₆·5H₂O: sometimes referred to as “OCP” below), has many superior functions to those of HA. For example, octacalcium phosphate is excellent in osteoconductivity, absorbability by osteoclasts, and dose-dependent promotion of differentiation of osteoblasts. Moreover, it has been reported that precursors of HA, amorphous calcium phosphate (Ca₃ (PO₄)₂·nH₂O) and calcium hydrogen phosphate (anhydrous calcium hydrogen phosphate (CaHPO₄) or calcium hydrogen phosphate dihydrate (CaHPO₄·2H₂O)), have similar properties to those of OCP. Therefore, HA precursors such as OCP, rather than HA, are expected as promising artificial bone materials.

However, to use OCP as an artificial bone material, OCP is very fragile and has low formability. To compensate for the low formability of OCP, a composite of OCP and a polymer material has been examined. For example, a composite of granules of OCP and collagen (sometimes referred to as “OCP/Col” below) is known. Although OCP/Col promotes the excellent osteoconductivity of OCP, when OCP/Col is used as an artificial bone material, the rate of loss of the artificial bone is slower than the regeneration rate of the bone in the process in which the artificial bone is lost because the artificial bone component is absorbed by the surrounding tissue and in which the bone regenerates by replacing the artificial bone. This is a property caused because the granules of OCP are not easily absorbed completely in the living body. It is a very important issue required in this field that the regenerated bone sufficiently replaces the artificial bone and that the bone defect is completely filled with the new bone.

PTL 1 by the present inventors discloses a bone regeneration material containing a dehydrothermally cross-linked material of a co-precipitate of octacalcium phosphate and gelatin. PTL 1 also discloses a method for producing a bone regeneration material including a step of adding dropwise or injecting an aqueous solution containing calcium to an aqueous solution containing gelatin and phosphorus and thus obtaining a co-precipitate of octacalcium phosphate and gelatin and a step of heating the co-precipitate to obtain a dehydrothermally cross-linked material. With the technique, a bone regeneration material which has excellent physical strength and has the same shape as the shape of the bone before the defect and which can be sufficiently replaced by the new bone is to be provided using the co-precipitate of OCP and gelatin (OCP/Gel).

### CITATION LIST

### Patent Literature

PTL 1: JP2011-234799A

### Non Patent Literature

NPL 1: Suzuki et al., Acta Biomaterialia, 88(2019), 514-526

### SUMMARY OF INVENTION

### Technical Problem

However, when the OCP/Gel is used under severe conditions, such as a large bone defect, there is a limit on the expression of the functions of the OCP/Gel, and a bone regeneration material having higher bone regeneration capacity has been desired.

The invention has been made under the circumstances, and an object thereof is to provide a bone-regeneration-promoting material having excellent bone regeneration capacity and a production method thereof.

### Solution to Problem

To solve the problem, the invention has the following aspects.

[1] A bone-regeneration-promoting material
containing a composite of octacalcium phosphate and a bioabsorbable polymer and
having edge dislocations introduced in the octacalcium phosphate,
wherein the total dislocation density of the octacalcium phosphate is 0.30×10¹⁷ m⁻² or more.

[2] The bone-regeneration-promoting material according to [1], wherein the bioabsorbable polymer is gelatin.

[3] The bone-regeneration-promoting material according to [1] or [2] which further contains organic molecules.

[4] The bone-regeneration-promoting material according to [3], wherein the organic molecules are of at least one of gelatin and an amino acid.

[5] The bone-regeneration-promoting material according to [4], wherein the amino acid is phosphorylated serine or phosphorylated threonine.

[6] A method for producing a bone-regeneration-promoting material including
a step of adding dropwise or injecting an aqueous solution containing one of phosphoric acid and calcium to an aqueous solution containing organic molecules and the other one of phosphates and calcium and thus obtaining a co-precipitate of octacalcium phosphate and the organic molecules,
a step of sizing the co-precipitate, and
a step of dispersing the co-precipitate in a solution containing a bioabsorbable polymer.

[7] The method for producing a bone-regeneration-promoting material according to [6], wherein the organic molecules are of at least one of gelatin and an amino acid.

[8] The method for producing a bone-regeneration-promoting material according to [7], wherein the amino acid is phosphorylated serine or phosphorylated threonine.

### Advantageous Effects of Invention

According to the invention, a bone-regeneration-promoting material having excellent bone regeneration capacity and a production method thereof can be provided.

### BRIEF DESCRIPTION OF DRAWINGS

FIG. 1 A figure showing the elution rates of c-OCP and w-OCP.

FIG. 2 A figure showing the bone regeneration capacity of c-OCP/Gel and w-OCP/Gel.

FIG. 3 A figure showing the concentrations of the inorganic phosphate ions eluted from c-OCP, P-ser-OCP, P-thr-OCP or w-OCP into the buffer.

FIG. 4 A figure show the ratios of the concentration of the inorganic phosphate ions eluted from c-OCP, P-ser-OCP or P-thr-OCP into the buffer to the concentration of the inorganic phosphate ions eluted from w-OCP into the buffer.

### DESCRIPTION OF EMBODIMENTS

The bone-regeneration-promoting material and the production method thereof according to the invention will be explained below with embodiments. The invention, however, is not limited to the following embodiments.

### [Bone-Regeneration-Promoting Material]

The bone-regeneration-promoting material of the embodiment is a material containing a composite of octacalcium phosphate (OCP) and a bioabsorbable polymer. Specifically, the bone-regeneration-promoting material of the embodiment is a material containing a composite of granules of a co-precipitate of octacalcium phosphate and organic molecules (c-OCP) and a bioabsorbable polymer.

The bone-regeneration-promoting material of the embodiment has edge dislocations introduced in the octacalcium phosphate, and the total dislocation density of the octacalcium phosphate is 0.30×10¹⁷ m⁻² or more. The total dislocation density is preferably 0.40×10¹⁷ m⁻² or more, more preferably 1.00×10¹⁷ m⁻² or more. When the total dislocation density is less than the lower limit value, the bone regeneration capacity of the bone-regeneration-promoting material deteriorates.

The total dislocation density of the composite can be measured by the following method.

The composite is observed with a high-resolution transmission electron microscope (HRTEM). The obtained HRTEM image is processed with fast Fourier transformation (FFT), and an FFT pattern is obtained.

In the obtained FFT pattern, the spots attributed to 002 or 030 of OCP are filtered, and an iFFT image (filtered HRETM image) is obtained by inverse FFT (iFFT) processing.

The number of edge dislocations observed in the obtained iFFT image is counted, and the dislocation density is measured. The average of measurement values from any three areas are regarded as the dislocation densities of one crystal, and the average value of two crystals is regarded as the average dislocation density of the sample.

The particle size of the granules is preferably 10 μm to 1000 μm, more preferably 300 μm to 500 μm. When the particle size of the granules is the lower limit value or less, the absorbability of the bone regeneration material in the living body due to the physicochemical solubility of the granules decreases. When the particle size of the granules is the upper limit value or more, the bone regeneration capacity of the obtained bone-regeneration-promoting material decreases.

Examples of the method for measuring the particle size of the granules include an observation with an optical microscope, an observation with a scanning electron microscope, an observation with a transmission electron microscope, centrifugal sedimentation, laser diffraction, dynamic light scattering, and the like.

The organic molecule content of the bone-regeneration-promoting material of the embodiment is preferably 0.00001 mass % to 30.0 mass %, more preferably 0.00005 mass % to 20.0 mass %, and further preferably 0.001 mass % to 12.0 mass %.

The ratio of the OCP and the bioabsorbable polymer in the bone-regeneration-promoting material of the embodiment is not particularly limited, but the ratio of the OCP with the bioabsorbable polymer as 1, by mass ratio, is preferably 0.1 to 9, more preferably 0.67 to 4. When the ratio of the OCP with the bioabsorbable polymer as 1 is less than 0.1, the bone regeneration capacity of the obtained bone-regeneration-promoting material deteriorates, and when the ratio exceeds 9, the shape-imparting property decreases.

The organic molecules in the embodiment are of a polymer which can introduce dislocations to the OCP. As the organic molecules, for example, a natural polymer (gelatin (acidic extraction or alkaline extraction), collagen, alginic acid, hyaluronic acid, chitosan, or the like), a synthetic polymer (polyacrylic acid, polyvinyl alcohol, polyethylene glycol, or the like), an amino acid (serine, threonine, phosphorylated serine, phosphorylated threonine, glycine, alanine, valine, leucine, isoleucine, asparagine, glutamine, cysteine, methionine, phenylalanine, tyrosine, tryptophan, proline, hydroxyproline, aspartic acid, glutamic acid, lysine, δ-hydroxylysine, arginine, histidine, or the like), or a peptide (polyserine, polythreonine, poly phosphorylated serin (poly O-Phospho-L-serine), poly phosphorylated threonine, or the like) can be widely used. The organic molecules can be widely selected in the range in which the effects of the invention are not inhibited.

The bioabsorbable polymer in the embodiment is a polymer which serves as a scaffold material of the composite for dispersing the OCP to that dislocations have been introduced and which can be absorbed in the living body. A bioabsorbable natural polymer (gelatin, collagen, alginic acid, hyaluronic acid, chitosan, or the like) or a bioabsorbable synthetic polymer (polylactic acid, a polylactic acid-glycolic acid copolymer, polycaprolactone, or the like) can be widely used. The bioabsorbable polymer can be widely selected in the range in which the effects of the invention are not inhibited.

In the embodiment, gelatin or an amino acid is preferably used as the organic molecules. One kind of gelatin and an amino acid may be used alone, or two or more kinds thereof may be used in combination.

The gelatin is not particularly limited. Generally, the gelatin is obtained by heat treating collagen. The gelatin may be commercial gelatin.

The collagen is not particularly limited. Examples thereof include collagen derived from porcine or bovine skin, bone, or tendon. Enzyme-solubilized collagen which has been solubilized with a protease (for example, pepsin or pronase) and from which telopeptides have been removed is preferable. The type of the collagen is preferably, for example, type I or type I+type III. Collagen is a living body-derived component and is thus highly safe, and in particular, enzyme-solubilized collagen is preferable because the allergenicity is low. The collagen may be commercial collagen.

The amino acid is preferably phosphorylated serine or phosphorylated threonine in view of the interaction with the OCP crystals.

In the embodiment, when gelatin is used, the bone-regeneration-promoting material is a composite of OCP and gelatin (OCP/Gel). The OCP/Gel is a composite in which gelatin and crystals of OCP are mixed, and the crystals of OCP are believed to be uniformly dispersed.

The phosphorus is not particularly limited as long as it is a compound which generates HPO₄²⁻ or PO₄³⁻ in an aqueous solution. Examples of such a compound include phosphates such as sodium hydrogen phosphate and ammonium phosphate and orthophosphoric acid.

The calcium is not particularly limited as long as it is a compound which generates Ca²⁺ in an aqueous solution. Examples of such a compound include calcium acetate, calcium chloride, and calcium nitrate.

The ratio of phosphorus and calcium is not particularly limited, but the ratio of phosphoric acid with calcium as 1, by mole ratio, is preferably 0.71 to 1.10, more preferably 0.73 to 1.00.

The bone-regeneration-promoting material of the embodiment may contain a dehydrothermally cross-linked material of the composite of the OCP and the bioabsorbable polymer. The dehydrothermally cross-linked material is a structure in which the bioabsorbable polymer forming the composite are mutually cross-linked by a dehydrocondensation reaction. The dehydrothermally cross-linked material has a cross-linked structure and thus is physically highly strong and is absorbed in the living body at an adequate rate.

The bone-regeneration-promoting material of the embodiment may appropriately contain another component or may be appropriately processed so that the bone-regeneration-promoting material is easily used for bone regeneration.

Because the bone-regeneration-promoting material of the embodiment contains a composite of octacalcium phosphate and a bioabsorbable polymer and has edge dislocations introduced in the octacalcium phosphate and because the total dislocation density of the octacalcium phosphate is 0.30×10¹⁷ m⁻² or more, the bone regeneration capacity is excellent.

### [Production Method of Bone-Regeneration-Promoting Material]

The method for producing a bone-regeneration-promoting material of the embodiment includes a step of adding dropwise or injecting an aqueous solution containing one of phosphoric acid and calcium to an aqueous solution containing organic molecules and the other one of phosphoric acid and calcium and thus obtaining a co-precipitate of octacalcium phosphate and the organic molecules (referred to as “the first step” below), a step of sizing the co-precipitate (referred to as “the second step” below), and a step of dispersing the sized co-precipitate in a solution containing a bioabsorbable polymer (referred to as “the third step” below).

In the first step, the pH values of the aqueous solution containing organic molecules and phosphoric acid and the aqueous solution containing organic molecules and calcium are preferably 4.5 to 7.5. A buffer component may be contained to avoid a change in the pH through mixing of the aqueous solution containing phosphoric acid or the aqueous solution containing calcium to the organic molecules. Moreover, at least one of the aqueous solution containing phosphoric acid and the aqueous solution containing calcium may contain another component. The other component may contain a bioabsorbable polymer.

The dropwise addition or the injection of the aqueous solution containing calcium to the aqueous solution containing organic molecules and phosphoric acid or the dropwise addition or the injection of the aqueous solution containing phosphoric acid to the aqueous solution containing organic molecules and calcium is conducted preferably at 50° C. to 80° C., more preferably at about 60° C. to 75° C. When the temperature is lower than 50° C. or exceeds 80° C., the OCP is not easily produced.

The “dropwise addition” here means that droplets of a solution are added to another solution. The “injection” means that a solution is added to another solution using a hollow tube such as a tube.

The dropwise addition or the injection is conducted while the aqueous solution containing organic molecules and phosphoric acid or the aqueous solution containing organic molecules and calcium is stirred. Without stirring, the OCP having a uniform particle size cannot be obtained.

The rate (mL/minute) of the dropwise addition or the injection is preferably 30 to 120, more preferably 35 to 82. When the rate is less than 30 or exceeds 120, the OCP is not easily produced.

In the first step, the mixing ratio of the aqueous solution containing phosphoric acid and the aqueous solution containing calcium is not particularly limited, but, with respect to the ratio of phosphoric acid and calcium, the ratio of phosphoric acid with calcium as 1, by mole ratio, is preferably 0.71 to 1.10, more preferably 0.73 to 1.00.

In the first step, the mixing ratio of the organic molecules to the aqueous solution containing phosphoric acid or the aqueous solution containing calcium is not particularly limited, but the organic molecule concentration in the aqueous solution is in the range of preferably 0.0004 mmol/L to 3.2 mmol/L, more preferably 0.004 mmol/L to 1.2 mmol/L. When the organic molecule concentration mixed in the aqueous solution containing phosphoric acid or the aqueous solution containing calcium is less than 0.0004 mmol/L, the amount of dislocations introduced to the obtained bone-regeneration-promoting material decreases. When the organic molecule concentration mixed in the aqueous solution containing phosphoric acid or the aqueous solution containing calcium exceeds 3.2 mmol/L, the crystallinity of the bone-regeneration-promoting material deteriorates considerably.

The organic molecules can be selected from those listed above.

In the second step, the co-precipitate of octacalcium phosphate and the organic molecules (c-OCP) obtained in the first step is recovered as a solid (granules). Examples of the solid include forms such as crystals and aggregates of crystals. The c-OCP precipitates as crystals or aggregates of crystals and thus can be recovered from the aqueous solution by filtration, drying, or the like.

The recovered solid (granules) may be washed with water, an organic solvent, or the like. The organic molecules may remain in the solid (granules) after washing.

The recovered granules of c-OCP are made uniform (sized). The method for making the granules uniform (sizing) is not particularly limited, but an example is a method for freeze-drying or naturally drying (air-drying) the granules and then pulverizing. The pulverizing method is not particularly limited, but is preferably a method for pulverizing the c-OCP using a mortar and a pestle or a mechanically pulverizing method. The mechanically pulverizing means is not particularly limited. An example thereof is means using a hard tissue pulverizer (beads shocker), a ball mill, or a crusher.

The particle size of the granules after the pulverization is generally 10 μm to 1000 μm, preferably 300 μm to 500 μm. When the particle size exceeds 1000 μm, the absorbability of the bone regeneration material in the living body due to the physicochemical solubility of the granules decreases.

In the third step, the sized granules (co-precipitate) are dispersed again in a solution containing a bioabsorbable polymer.

The amount of the granules added to the solution containing a bioabsorbable polymer is represented by the ratio of the c-OCP and the bioabsorbable polymer and is not particularly limited, but the ratio of the c-OCP with the bioabsorbable polymer as 1, by mass ratio, is in the range of preferably 0.1 to 9, more preferably 0.67 to 4. When the ratio of the c-OCP with the bioabsorbable polymer as 1 is less than 0.1, the bone regeneration capacity of the obtained bone-regeneration-promoting material deteriorates, and when the ratio exceeds 9, the shape-imparting property decreases.

To disperse the sized granules in the solution containing a bioabsorbable polymer again, the solution containing a bioabsorbable polymer and the granules are preferably stirred.

After the completion of stirring, the composite of the c-OCP and the bioabsorbable polymer is recovered. Then, the composite is freeze-dried or naturally dried (air-dried), and thus the bone-regeneration-promoting material of the embodiment is obtained.

In this regard, the method for producing a bone-regeneration-promoting material of the embodiment may include a step of obtaining a cross-linked material by dehydrothermal cross-linking by cross-linking the bioabsorbable polymer in the composite through dehydrothermal condensation, chemical cross-linking by cross-linking through a covalent bond by a chemical reaction, or cross-linking by irradiation with electron beam or radiation. The dehydrothermally cross-linked material is obtained by heating the OCP/Gel. The heating treatment is conducted at a temperature of 50° C. to 200° C., preferably 100° C. to 150° C., for 3 hours to 240 hours, preferably 24 hours to 100 hours.

The dehydrothermally cross-linked material is preferably obtained by heating the composite of the c-OCP and the bioabsorbable polymer under reduced pressure. The reduced pressure condition is not particularly limited, but is, for example, 200 Pa or less, preferably 133 Pa or less.

The dehydrothermally cross-linked material is more preferably obtained by drying the composite of the c-OCP and the bioabsorbable polymer and then heating under reduced pressure. The drying method is not particularly limited, but is, for example, a freeze-drying method or a natural-drying method (air-drying). The drying step may be made efficient by leaving the composite of the c-OCP and the bioabsorbable polymer still before drying and then, for example, removing the supernatant to appropriately reduce the water content.

In the method for producing a bone-regeneration-promoting material of the embodiment, the granules may be directly used for the bone-regeneration-promoting material, or the method may include a step of appropriately adding another component for easy use for bone regeneration or include a step of processing such as forming.

The bone-regeneration-promoting material of the invention is appropriately formed depending on the shape of the bone defect part and implanted in the bone defect part after sterilization treatment by irradiation with electron beam, high-pressure steam sterilization, or the like. The high-pressure steam sterilization, however, affects the crystal phase of the OCP, and thus in this case, the application site of the bone defect is taken into consideration.

Although embodiments of the invention have been explained above, the invention is not limited to the embodiments, and various changes can be made.

### EXAMPLES

The effects of the invention will be further clarified below with Examples and Comparative Example. Here, the invention is not limited only to the Examples below and can be carried out with an appropriate change in the range in which the gist thereof is not changed.

### Example 1

By the wet method based on (Suzuki et al. Tohoku J. Exp. Med. 164 (1991) 37-50), a co-precipitate of OCP and organic molecules, namely c-OCP, was synthesized.

An aqueous 0.04 mol/L calcium acetate solution in a volume of 1 L was added dropwise at 65° C. over 15 minutes to 1 L of an aqueous phosphate solution (an aqueous 0.04 mol/L sodium dihydrogen phosphate dihydrate solution, pH 4.5 at room temperature) containing 0.2 mmol/L gelatin (manufactured by Sigma-Aldrich, Type A Gelatin (derived from porcine skin, number-average molecular weight (Mn): 50000 to 100000)) as organic molecules and further mixed at 70° C. for several minutes, and a precipitate (co-precipitate) was formed. The supernatant was removed, and the granules of c-OCP were recovered as aggregates of crystals. The recovered granules of c-OCP were dried at 105° C., pulverized with a mortar and a pestle and caused to pass through a 32 mesh to 48 mesh filter, and thus granules having a diameter of 300 μm to 500 μm were obtained.

Subsequently, an aqueous solution (aqueous gelatin solution) containing 3 mass % gelatin (manufactured by Sigma-Aldrich, Type A Gelatin (derived from porcine skin, number-average molecular weight (Mn): 50000 to 100000)) as a bioabsorbable polymer was prepared. The granules of c-OCP were dispersed in the aqueous gelatin solution in such a manner that the c-OCP content became 46 mass % of the total mass of the c-OCP/Gel. The suspension of the granules was transferred to a polypropylene container, mixed at 4° C. for galating the gelatin and then frozen to −20° C. for freeze-drying, and thus a bone-regeneration-promoting material containing a composite of c-OCP and gelatin (c-OCP/Gel) was obtained.

### Example 2

P-ser-OCP was synthesized in the same manner as in Example 1 except that an aqueous phosphate solution containing 0.2 mmol/L phosphorylated serine (P-ser) was used instead of the aqueous phosphate solution containing gelatin.

### Example 3

P-thr-OCP was synthesized in the same manner as in Example 1 except that an aqueous phosphate solution containing 0.2 mmol/L phosphorylated threonine (P-thr) was used instead of the aqueous phosphate solution containing gelatin.

### Comparative Example

By the wet method based on (Suzuki et al. Tohoku J. Exp. Med. 164 (1991) 37-50), w-OCP was synthesized from a solution which contained a phosphate and which did not contain any third component such as organic molecules and an aqueous solution containing calcium.

An aqueous phosphate solution (an aqueous 0.04 mol/L sodium dihydrogen phosphate dihydrate solution, pH 4.5 at room temperature) in a volume of 1 L was added dropwise at 65° C. over 15 minutes to 1 L of an aqueous 0.04 mol/L calcium acetate solution and further mixed at 70° C. for several minutes, and a precipitate was formed. The supernatant was removed, and the granules of w-OCP were recovered as aggregates of crystals. The recovered granules of w-OCP were dried at 105° C., pulverized with a mortar and a pestle and caused to pass through a 32 mesh to 48 mesh filter, and thus granules having a diameter of 300 μm to 500 μm were obtained.

Subsequently, an aqueous solution (aqueous gelatin solution) containing 3 mass % gelatin (manufactured by Sigma-Aldrich, Type A Gelatin (derived from porcine skin, number-average molecular weight (Mn): 50000 to 100000)) as a bioabsorbable polymer was prepared. The obtained granules were dispersed in the aqueous gelatin solution. The granules of w-OCP were dispersed in the aqueous gelatin solution in such a manner that the w-OCP content became 46 mass % of the total mass of the w-OCP/Gel. The dispersion of the granules was transferred to a polypropylene container, mixed at 4° C. for galating the gelatin, then frozen to −20° C., and then freeze-dried, and thus a bone-regeneration-promoting material containing a composite of OCP and gelatin (w-OCP/Gel) was obtained.

### (Measurement of Dislocation Densities of OCP)

The total dislocation densities of the OCP of Examples 1 to 3 and the Comparative Example were measured by the following method.

Each OCP was observed with a high-resolution transmission electron microscope (HRTEM). The obtained HRTEM image was processed with fast Fourier transformation (FFT), and an FFT pattern was obtained.

In the obtained FFT pattern, the spots attributed to 002 or 030 of OCP were filtered, and an iFFT image (filtered HRETM image) was obtained by inverse FFT (iFFT) processing.

The number of edge dislocations observed in the obtained iFFT image was counted, and the dislocation density was measured. The average of measurement values from any three areas were regarded as the dislocation densities of one crystal, and the average value of two crystals was regarded as the average dislocation density of the sample. The results are shown in Table 1.

TABLE 1

| | | Dislocation | Dislocation | Total |
|---|---|---|---|---|
| | | Density of | Density of | Dislocation |
| | | (002) | (030) | Density |
| | Sample | [×10¹⁶ m⁻²] | [×10¹⁶ m⁻²] | [×10¹⁷ m⁻²] |
| Example 1 | c-OCP | 8.05 | 3.99 | 1.21 |
| Example 2 | P-ser-OCP | 0.2 | 4.21 | 0.44 |
| Example 3 | P-thr-OCP | 1.57 | 4.57 | 0.62 |
| Comparative | w-OCP | 0.94 | 1.05 | 0.2 |
| Example | | | | |

From the results shown in Table 1, it was found that the total dislocation densities of the c-OCP/Gel of Example 1, the P-ser-OCP of Example 2, and the P-thr-OCP of Example 3 were higher than that of the w-OCP of Comparative Example.

### (Measurement of Elution Rates of OCP)

The c-OCP obtained in Example 1 or the w-OCP obtained in Comparative Example was immersed in a 150 mmol/L tris hydroxymethyl aminomethane-hydrochloride buffer adjusted to pH 7.4 at 37° C. for 30 seconds, 60 seconds, 180 seconds, or 300 seconds. The solid liquid ratio was solid/liquid=1 mg/1 mL.

The calcium ion (Ca²⁺) concentrations of the supernatants at the immersion times were measured using a kit (Calcium E test Wako), and the amounts of the eluted OCP were calculated.

From the approximate curve showing the relation between the immersion time and the amount of the eluted OCP, the elution rates of the immersion times were calculated. The elution rates were normalized with the surface area of the c-OCP or the w-OCP. The change in the surface area with the elution was estimated using the following equation (1).

$$A = {m}_{0} \cdot S \cdot {( {m}_{t} / {m}_{0} )}^{2 / 3} \qquad ( 1 )$$

In the above equation (1), A is the surface area of the sample at the immersion time t, and m₀ is the mass of the sample at t=0. S is the specific surface area of the sample, and mₜ is the mass of the sample at the immersion time t.

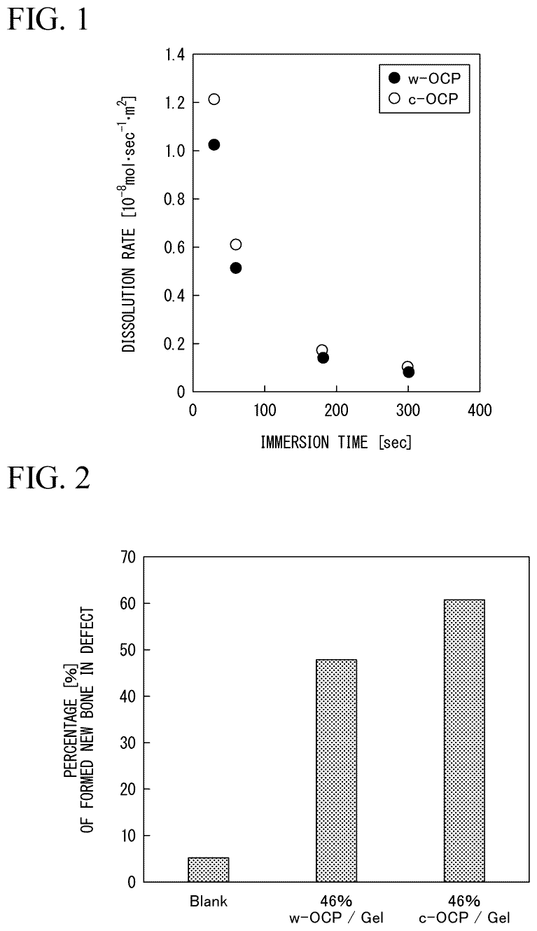

The results are shown in FIG. 1.

From the results shown in FIG. 1, it was found that the elution rate of the c-OCP was higher than that of the w-OCP.

### (Evaluation of Bone Regeneration Capacity of OCP)

The c-OCP/Gel obtained in Example 1 or the w-OCP/Gel obtained in Comparative Example was formed into a disk having a diameter of 9 mm and a thickness of 1 mm and vacuum heated at 150° C. for 24 hours to cross-link the gelatin.

A critical sized bone defect with a diameter of 9 mm was created in the calvaria of Wistar rats (male, 12 weeks old), and the disk composed of the c-OCP/Gel or the disk composed of the w-OCP/Gel was implanted in the critical sized bone defect.

The calvaria was recovered eight weeks after the implantation, and a thin slice was produced after decalcification. The slice was stained with hematoxylin and eosin (HE), and the new bone tissue formed in the defect area was observed under an optical microscope.

By image analysis of the obtained HE staining image, the bone defect area and the new bone area were measured, and the percentage (%) of the formed new bone in the defect was calculated.

The results are shown in FIG. 2.

In FIG. 2, “Blank” shows the results of an observation of the bone regeneration with the bone defect only, namely without the implantation of the bone-regeneration-promoting material in the bone defect area. “Blank” shows that, regarding the bone defect, bone formation does not actually occur in the animal model with the bone defect with a size that cannot be self-repaired (critical sized bone defect) without implantation of the bone-regeneration-promoting material.

From the results shown in FIG. 2, it was found that the c-OCP/Gel promotes bone regeneration more than the w-OCP/Gel.

### (Evaluation of Solubility of OCP in Buffer)

c-OCP synthesized in the same manner as in Example 1 above, P-ser-OCP synthesized in the same manner as in Example 2, P-thr-OCP synthesized in the same manner as in Example 3 and w-OCP synthesized in the same manner as in Comparative Example were prepared each in an amount of 5 mg. The composites each in an amount of 5 mg were immersed in 5 mL of a 150 mmol/L tris hydroxymethyl aminomethane-hydrochloride (Tris-HCl) buffer (containing 0.5 mmol/L calcium ion and 0.5 mmol/L inorganic phosphate ion, pH 7.4, 37° C.) and stirred by inverting.

The supernatants were recovered using centrifugation 24 hours after starting the immersion.

The concentrations of the inorganic phosphate ions contained in the recovered supernatants were measured with Phospha C-Test Wako (manufactured by FUJIFILM Wako Pure Chemical Corporation).

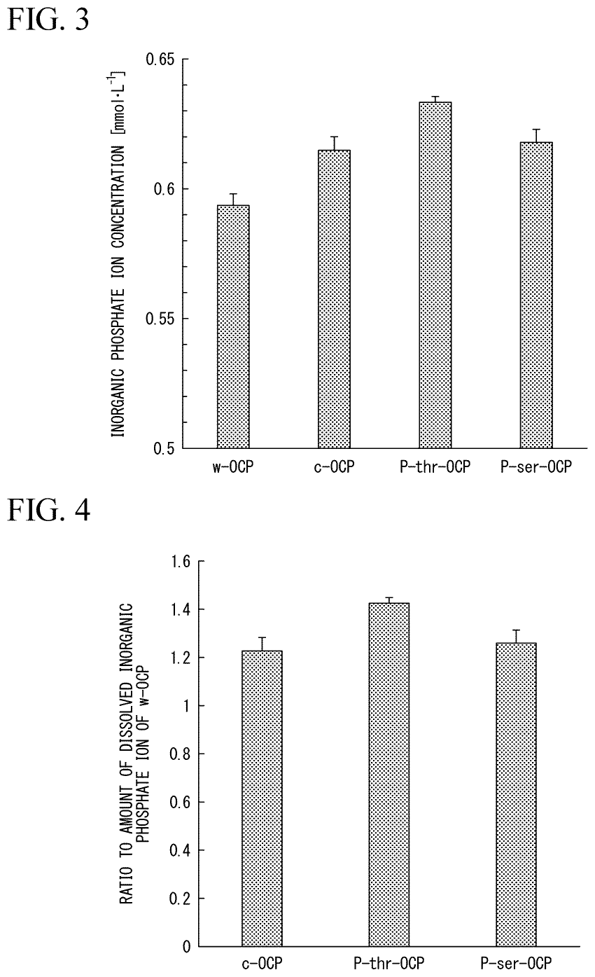

The concentrations of the inorganic phosphate ions eluted from the composites into the buffer are shown in FIG. 3. Moreover, the ratios of the concentration of the inorganic phosphate ions eluted from c-OCP, P-ser-OCP, or P-thr-OCP into the buffer to the concentration of the inorganic phosphate ions eluted from w-OCP into the buffer are shown in FIG. 4.

From the results shown in FIGS. 3 and 4, it could be found that OCP into which dislocations were introduced at a high density (c-OCP, P-ser-OCP or P-thr-OCP) had a higher inorganic phosphate ion concentration and improved self-solubility compared to w-OCP having a low dislocation density without any treatment for increasing the dislocation density.

It is believed that the OCP activities of P-ser-OCP and P-thr-OCP will increase by introducing dislocations as c-OCP.

### INDUSTRIAL APPLICABILITY

According to the bone-regeneration-promoting material and the production method thereof of the invention, a bone-regeneration-promoting material having excellent bone regeneration capacity is obtained.

1. A bone-regeneration-promoting material,
comprising a composite of octacalcium phosphate and a bioabsorbable polymer and
having edge dislocations introduced in the octacalcium phosphate,
wherein a total dislocation density of the octacalcium phosphate is 0.30×10¹⁷ m⁻² or more.

2. The bone-regeneration-promoting material according to claim 1, wherein the bioabsorbable polymer is gelatin.

3. The bone-regeneration-promoting material according to claim 1, further comprising organic molecules.

4. The bone-regeneration-promoting material according to claim 3, wherein the organic molecules are of at least one molecule of gelatin and an amino acid.

5. The bone-regeneration-promoting material according to claim 4, wherein the amino acid is phosphorylated serine or phosphorylated threonine.

6. A method for producing a bone-regeneration-promoting material comprising:
a step of adding dropwise or injecting an aqueous solution containing one of phosphates and calcium to an aqueous solution containing organic molecules and the other one of phosphoric acid and calcium and thus obtaining a co-precipitate of octacalcium phosphate and the organic molecules;
a step of sizing the co-precipitate; and
a step of dispersing the co-precipitate in a solution containing a bioabsorbable polymer.

7. The method for producing a bone-regeneration-promoting material according to claim 6, wherein the organic molecules are of at least one molecule of gelatin and an amino acid.

8. The method for producing a bone-regeneration-promoting material according to claim 7, wherein the amino acid is phosphorylated serine or phosphorylated threonine.

9. The bone-regeneration-promoting material according to claim 2, further comprising organic molecules.
